(19) United States
(12) Patent Application Publication
(10) Pub. No.: US 2010/0105998 A1
(43) Pub. Date: Apr. 29, 2010

(54) METHOD AND APPARATUS FOR SPECTROPHOTOMETRIC BASED OXIMETRY OF SPINAL TISSUE

(75) Inventor: Paul Benni, Guilford, CT (US)

Correspondence Address: O''Shea Getz P.C., 1500 MAIN ST. SUITE 912, SPRINGFIELD, MA, 01115, US

(73) Assignee: CAS MEDICAL SYSTEMS, INC., Branford, CT (US)

(21) Appl. No.: 12/607,648

(22) Filed: Oct. 28, 2009

Related U.S. Application Data
(60) Provisional application No. 61/109,053, filed on Oct. 28, 2008.

(51) Int. Cl. A61B 5/1455

(52) U.S. Cl. 600/340

(57) ABSTRACT

A near infrared spectrophotometric sensor for non-invasive monitoring of blood oxygenation levels in a subject's spinal cord tissue and spinal cord blood vessels is provided. The sensor includes at least one light source and at least one light detector. The light source is operative to emit near infrared light signals of a plurality of different wavelengths. The light detector is operative to sense light signals emitted from the light source and passed through the subject's spinal tissue, and to produce a sensor signal representative of the sensed light signals The light source is separated from the light detector by a distance representative of a distance from a first vertebrae structure of a human spine to a second vertebrae structure of the human spine, to permit alignment of the light source and detector with the first and second vertebrae structure.

This application claims the benefit of U.S. Provisional Patent Application Ser. No. 61/109,053, filed Oct. 28, 2008, which is hereby incorporated in its entirety.

### BACKGROUND OF THE INVENTION

1. Technical Field

This invention relates to methods and apparatus for non-invasively determining biological tissue oxygenation utilizing near-infrared spectroscopy (NIRS) techniques in general, and to methods and apparatus for sensing the oxygen saturation level of a subject's spine tissue in particular.

2. Background Information

Near-infrared spectroscopy is an optical spectrophotometric method that can be used to continuously monitor tissue oxygenation. The NIRS method is based on the principle that light in the near-infrared range (700 nm to 1,000 nm) can pass easily through skin, bone and other tissues where it encounters hemoglobin located mainly within micro-circulation passages; e.g., capillaries, arterioles, and venuoles. Hemoglobin exposed to light in the near-infrared range has specific absorption spectra that varies depending on its oxidation state; i.e., oxyhemoglobin (HbO₂) and deoxyhemoglobin (Hb) each act as a distinct chromophore. By using light sources that transmit near-infrared light at specific different wavelengths, and measuring changes in transmitted or reflected light attenuation, concentration changes of the oxyhemoglobin (HbO₂) and deoxyhemoglobin (Hb) can be monitored.

NIRS type sensors typically include at least one light source and one or more light detectors for detecting reflected or transmitted light. The light signal is created and sensed in cooperation with a NIRS system that includes a processor and an algorithm for processing signals and the data contained therein. PCT Publication No. WO 2008/118216 and U.S. Pat. No. 7,047,054, which are commonly assigned with the present application to CAS Medical Systems, Inc. of Branford, Conn., disclose examples of such a sensor operable to sense cerebral tissue oxygenation. Light sources such as light emitting diodes (LEDs) or laser diodes that produce light emissions in the wavelength range of 700-1000 nm are typically used. A photodiode or other light detector is used to detect light reflected from or passed through the tissue being examined. The NIRS system cooperates with the light source(s) and the light detectors to create, detect and analyze the signals in terms of their intensity and wave properties. U.S. Pat. No. 6,456,862, and U.S. Pat. No. 7,072,701, both of which are commonly assigned to CAS Medical Systems, Inc., of Branford, Conn., disclose a methodology for analyzing such signals. U.S. Pat. Nos. 6,456,862, 7,047,054, and 7,072,701 and PCT Publication No. WO 2008/118216 are hereby incorporated by reference in their entirety.

The light emanating from the light source may be described as traveling along a “mean optical path” through the tissue under examination. The “mean optical path” represents an idealized path traveled by a predominant number of photons emanating from the light source and sensed by the detector, recognizing however that not all photons emanating from the light source will travel the mean optical path. The length of the mean optical path and the depth from the surface reached by the path are a function of the separation distance between the light source and the light detector and the geometry of the path. Several sources of research in NIRS technology provide that the mean optical path follows a “banana-shaped” path.

It is known that spinal tissue ischemia can result in neurologic sequelae. The ability to continually monitor spinal column oxygenation levels would, therefore, be particularly valuable.

What is needed, therefore, is NIRS device that can non-invasively determining the level of oxygen saturation within the spinal cord tissue of a subject.

### DISCLOSURE OF THE INVENTION

According to an aspect of the present invention, a near infrared spectrophotometric sensor for non-invasive monitoring of blood oxygenation levels in a subject's spinal cord tissue and spinal cord blood vessels is provided. The sensor includes at least one light source and at least one light detector. The light source is operative to emit near infrared light signals of a plurality of different wavelengths. The light detector is operative to sense light signals emitted from the light source and passed through the subject's spinal tissue, and to produce a sensor signal representative of the sensed light signals. The light source is separated from the light detector by a distance representative of a distance from a first vertebrae structure of a human spine to a second vertebrae structure of the human spine, to permit alignment of the light source and detector with the first and second vertebrae structure.

According to another aspect of the present invention, a near infrared spectrophotometric system for non-invasive monitoring of blood oxygenation levels in a subject's spinal cord tissue and spinal cord blood vessels is provided. The NIRS system includes one or more NIRS sensors and a processor. Each sensor has at least one light source and at least one light detector. The light source is operative to emit near infrared light signals of a plurality of different wavelengths. The light detector is operative to sense light signals emitted from the light source and passed through the subject's spinal tissue, and to produce a sensor signal representative of the sensed light signals. The light source is separated from the light detector by a distance representative of a distance from a first vertebrae structure of a human spine to a second vertebrae structure of the human spine, to permit alignment of the light source and detector with the first and second vertebrae structure. The processor is adapted to produce signals from the light source and receive sensor signals from the light detector, and to analyze such sensors signals to determine the blood oxygenation level within the subject's spinal cord tissue and spinal cord blood vessels.

According to another aspect of the present invention, a method for non-invasively monitoring blood oxygenation levels in a subject's spinal cord tissue and spinal cord blood vessels is provided. The method includes the steps of: a) providing at least one light source operative to emit near infrared light signals of a plurality of different wavelengths; b) aligning the light source with a first vertebrae structure of the subject; c) providing at least one light detector operative to sense light signals emitted from the light source and passed through the subject's spinal tissue, and produce a sensor signal representative of the sensed light signals; d) aligning the light detector with a second vertebrae structure of the subject; e) introducing the near infrared light signals into the subject from the light source in a manner such that light signals travel through the first vertebrae structure, pass through spinal cord tissue and spinal cord blood vessels, and pass through the second vertebrae structure; f) detecting light passing through the second vertebrae structure using the light detector, and producing sensor signals representative of such detected light; and g) processing the sensor signals to obtain data relating to the blood oxygenation level of the subject's spinal cord tissue and spinal cord blood vessels.

These and other features and advantages of the present invention will become apparent in light of the drawings and detailed description of the present invention provided below.

### BRIEF DESCRIPTION OF THE DRAWINGS

FIG. 1 shows a diagrammatic view of NIRS system, including a single NIRS spine sensor disposed on a subject's back.

FIG. 2 shows a diagrammatic view of a NIRS system, including a plurality of NIRS spine sensors disposed on a subject's back.

FIG. 3 is a simplified diagrammatic, exploded representation of an example of the type of NIRS sensor assembly that can be used with the present invention.

FIG. 4 is a cross-sectional diagrammatic view of the type of sensor shown in FIG. 3.

FIG. 5 is a cross-sectional diagrammatic view of a sensor similar to that shown in FIG. 3, with only a single detector.

FIG. 6 is a diagrammatic representation of a spinal column, illustrating a present invention sensor positioned to sense spinal tissue disposed within the spinal canal.

### DETAILED DESCRIPTION OF THE INVENTION

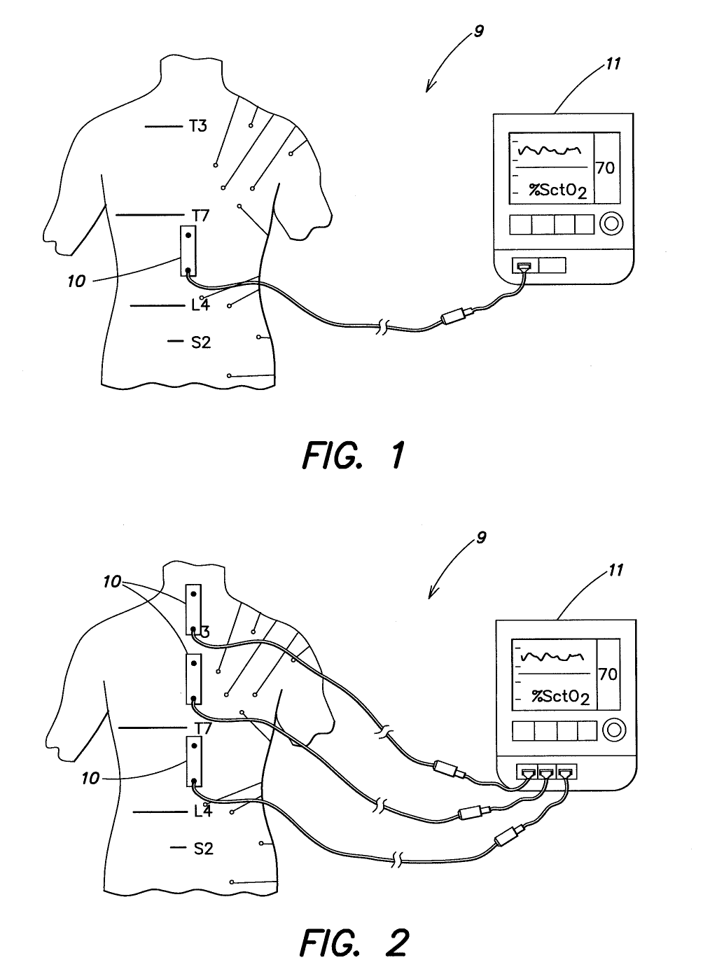

Referring now to the drawings, the present invention near infra-red spectroscopy (NIRS) system 9 includes one or more NIRS sensors 10 connected to a processor portion 11 of a NIRS system 9 (see FIGS. 1 and 2).

The NIRS system processor 11 is adapted to provide signals to, and receive signals from, one or more of the NIRS sensors 10. The processor 11 includes a central processing unit (CPU) adapted (e.g., programmed) to selectively perform the functions necessary to perform the present analysis of spinal tissue as described herein. It should be noted that the functionality of processor 11 may be implemented using hardware, software, firmware, or a combination thereof. A person skilled in the art would be able to program the processing unit to perform the functionality described herein without undue experimentation. Examples of acceptable NIRS systems are described in U.S. Pat. Nos. 6,456,862 and 7,072,701, which patents were incorporated by reference above. The algorithms (including the processors adapted to utilize these algorithms) described in these patents are examples of acceptable NIRS algorithms that can be adapted for use according to the disclosures of the present invention system. The present NIRS sensor 10 is not, however, limited to use with any particular NIRS system.

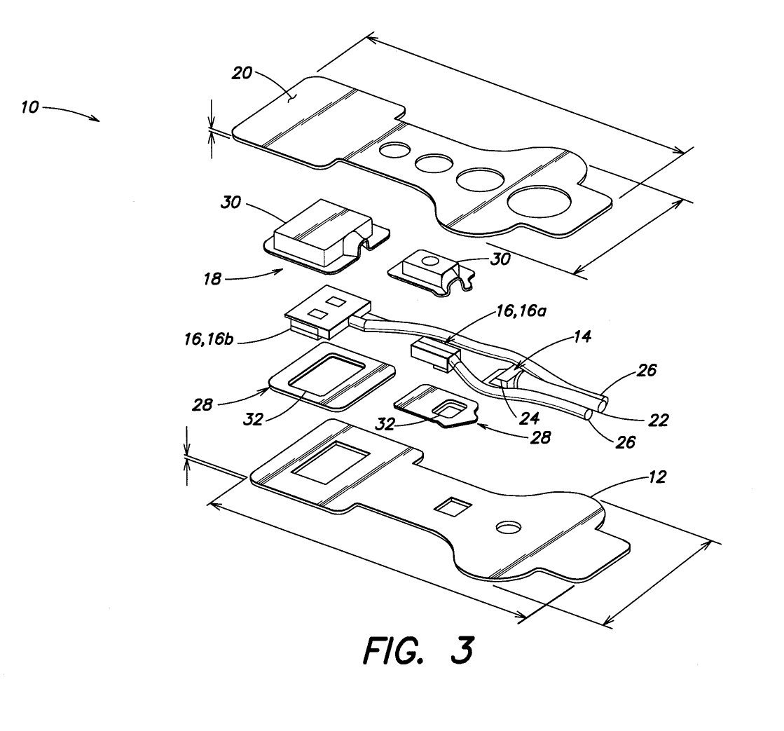

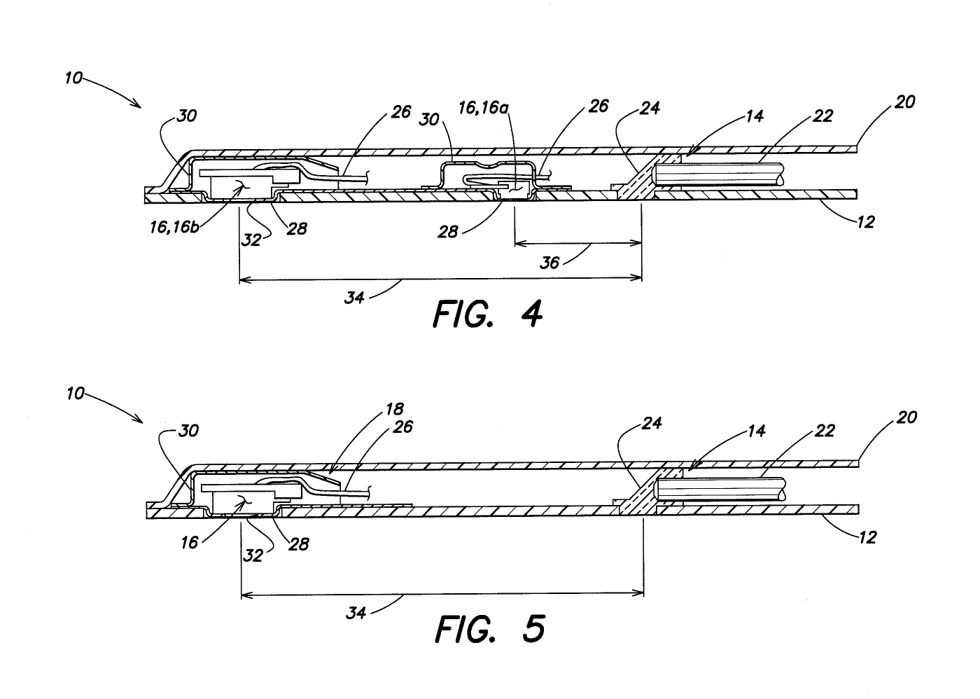

An embodiment of a NIRS sensor assembly 10 is shown in FIGS. 3-5. The NIRS sensor assembly includes a pad 12, at least one light source 14, at least one light detector 16, at least one detector housing 18, electromagnetic interference (EMI) shielding, and a cover 20. In those embodiments of the present sensor assembly 10 that include more than one light detector 16 (e.g., detectors 16a, 16b in FIGS. 3 and 4), the present invention may include a plurality of detector housings. An example of an acceptable NIRS sensor 10 is described in PCT Publication No. WO 2008/118216. The present application is not, however, limited to the NIRS sensor described in the aforesaid PCT publication.

The sensor light source 14 is selectively operable to guide or emit infrared light (i.e., light in wavelength range of about 700 nm to about 1,000 nm). As stated above, infrared light provides particular utility in determining tissue oxygenation because hemoglobin exposed to light in the near-infrared range has specific absorption spectra that varies depending on its oxidation state; i.e., oxyhemoglobin (HbO₂) and deoxyhemoglobin (Hb) each act as a distinct chromophore. In alternative embodiments, however, there may be utility in examining blood metabolites that are best examined with a light outside the infrared range; e.g., in the visible light range between 400 nm and 700 nm, such as red light at 650 nm, or green light at 510 nm, or both visible and infrared light combinations, etc. In those applications, a light source 14 may be utilized that emits or guides light outside the infrared range.

In the sensor embodiment shown in FIGS. 3-5, the light source 14 is an assembly that includes a fiber optic light guide 22 and a light redirecting prism 24. One end of the fiber optic guide 22 is optically connected to the prism 24. The other end of the fiber optic guide 22 connects directly or indirectly to the NIRS system 9. In alternative embodiments, the light source 14 may employ one or more LEDs mounted within the sensor assembly.

The light detector(s) 16 includes a light responsive transducer such as a photodiode that is operative to sense light intensity derived from light emitted by the light source 14 after such light passes through the subject's body. The light detectors 16 are electrically connected to the NIRS system 9 to enable the output of the light detectors 16 be communicated to the NIRS system 9. In a preferred embodiment, one or more EMI shielded cables 26 connect the light detectors 16 to the NIRS system 9.

The detector housing 18 includes a base 28 and a cap 30 that together define an internal cavity, which cavity is sized to enclose a light detector 16 at least partially covered with shielding (and other materials as applicable). The base 28 and the cap 30 may be hinged together or they may be separable. The base 28 includes a well that is sized to receive at least a portion of the light detector 16, and the cap 30 is sized to receive the remainder of the light detector 16 not received within the base well. The base well 28 includes a window panel 32 that consists of an optically transparent material that allows light to pass there through and be sensed by the light detector 16. The base 28 and the cap 30 may be made out of the same material or different materials.

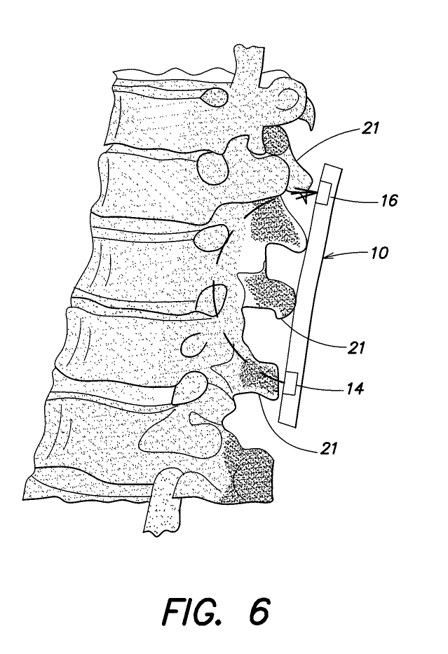

The light source 14 is separated from the detector(s) 16 by a defined distance 34, 36, respectively chosen so that the detector 16 and the light source 14 align with vertebrae structure 21 (e.g., spinous process, lamina, etc., see FIG. 6) of the subject. Separating the light source 14 and detector 16 a predetermined distance that substantially aligns each of the light source 14 and a detector 16 with a vertebrae structure 21 permits the sensor 10 to use vertebrae structures 21 as light guides into and out of the spinal canal; e.g., emitted light from the light source 14 travels through an aligned vertebrae structure 21 and into the spinal canal, through the spinal cord tissue, and subsequently out of a second aligned vertebrae structure 21 where it is sensed by the aligned detector 16. One of the significant advantages provided by the present invention light source—detector spacing is that the light signal traveling through the path provided by the vertebrae structure 21 experiences substantially lower attenuation than it would if it were traveling through the adjacent tissue; e.g., tissue containing blood. As a result of the lower attenuation, the sensor 10 is able to interrogate tissue (e.g., the spinal cord and associated blood vessels) located at a depth that would be practically speaking inaccessible using a conventional NIRS sensor; i.e., one with light source—detector separation distances that are acceptable for cerebral or organ interrogation.

Under the present invention, the light signal interrogation depth can be at least equal to half the separation distance 34 between the light source 14 and the detector 16 or preferably greater with the use of vertebrate structure as a light guide. An example of a light source/detector separation distance that is acceptable for spinal cord interrogation of most adults is approximately sixty-five millimeters (65 mm) A NIRS sensor 10 having a light source—detector separation distance 34 that is approximately sixty-five millimeters (65 mm) will not work effectively in a cerebral or organ sensing application of most adults because of an undesirable signal to noise ratio. The present invention is not limited to the aforesaid source—detector separation distance. On the contrary, as indicated above the source—detector separation distance is chosen so that the light source 14 and the detector 16 align with vertebrae structure 21 of the subject. Young/small adult, adolescent, or pediatric subjects may utilize a plurality of different source—detector separation distances. In addition, the spacing of vertebrae structure within a particular subject will likely vary in different regions of the spine; e.g., a sensor for use in the cervical region may use a source—detector spacing that is less than the source—detector spacing of a sensor used in the lumbar region of the same subject.

In the operation of the present invention, one or more NIRS sensors 10 are placed in contact with the skin on the subject's back, positioned along her spine. As shown in FIGS. 1, 2, and 4, the one or more NIRS sensors 10 are operable to be attached to, and aligned with vertebrae structure of the subject in the cervical, thoracic, lumbar, or pelvic spinal regions. Once positioned, the one or more sensors 10 are selectively actuated via signal control from the processor 11 and near infrared light signals are introduced into the subject's body tissue from the light source 14 of each sensor 10. The light initially passes through a first vertebrae structure 21 aligned with the light source 14, subsequently travels through the spinal cord tissue, and finally travels through a second vertebrae structure 21, where it is detected by a light detector 16 aligned with the second vertebrae structure 21. The light detector 16 produces signals representative of the detected light, which signals are relayed back to the NIRS system processor 11. The processor 11, which is adapted to the spinal tissue interrogation application, processes the signals to obtain data relating to the blood oxygenation level of the subject's body tissue; e.g., spinal cord tissue and spinal cord blood vessels. The data can be displayed in a variety of different modes (numeric, graphical, etc.) for the end-user's review.

Since many changes and variations of the disclosed embodiment of the invention may be made without departing from the inventive concept, it is not intended to limit the invention otherwise than as required by the appended claims.

What is claimed is:

1. A near infrared spectrophotometric sensor for non-invasive monitoring of blood oxygenation levels in a subject's spinal cord tissue and spinal cord blood vessels, said sensor comprising:
at least one light source operative to emit near infrared light signals of a plurality of different wavelengths;
at least one light detector operative to sense light signals emitted from the light source and passed through the subject's spinal tissue, and produce a sensor signal representative of the sensed light signals;
wherein the light source is separated from the light detector by a distance representative of a distance from a first vertebrae structure of a human spine to a second vertebrae structure of the human spine, to permit alignment of the light source and detector with the first and second vertebrae structure.

2. The sensor of claim 1, wherein the light source includes a fiber optic light guide and a light redirecting prism, and a single light detector.

3. The sensor of claim 2, wherein the light detector is separated from the light source by about sixty-five millimeters.

4. The sensor of claim 1 wherein the distance separating the light source from the light detector is based on a distance representative of a distance between first and second vertebrae structures located within a lumbar region of the human spine.

5. The sensor of claim 1 wherein the distance separating the light source from the light detector is based on a distance representative of a distance between first and second vertebrae structures located within a cervical region of the human spine.

6. The sensor of claim 1 wherein the distance separating the light source from the light detector is based on a distance representative of a distance between first and second vertebrae structures located within a thoracic region of the human spine.

7. A near infrared spectrophotometric system for non-invasive monitoring of blood oxygenation levels in a subject's spinal cord tissue and spinal cord blood vessels, said system comprising:
one or more sensors, each sensor having at least one light source operative to emit near infrared light signals of a plurality of different wavelengths, and at least one light detector operative to sense light signals emitted from the light source and passing through the subject's spinal tissue, and to produce a sensor signal representative of the sensed light signals, and wherein the light source is separated from the light detector by a distance representative of a distance from a first vertebrae structure of a human spine to a second vertebrae structure of the human spine, to permit alignment of the light source and detector with the first and second vertebrae structure; and
a processor adapted to produce signals from the light source and receive sensor signals from the light detector, and to analyze such sensors signals to determine the blood oxygenation level within the subject's spinal cord tissue and spinal cord blood vessels.

8. A method for non-invasively monitoring blood oxygenation levels in a subject's spinal cord tissue and spinal cord blood vessels, comprising the steps of:
providing at least one light source operative to emit near infrared light signals of a plurality of different wavelengths;
aligning the light source with a first vertebrae structure of the subject;
providing at least one light detector operative to sense light signals emitted from the light source and passed through the subject's spinal tissue, and produce a sensor signal representative of the sensed light signals;
aligning the light detector with a second vertebrae structure of the subject;
introducing the near infrared light signals into the subject from the light source in a manner such that light signals travel through the first vertebrae structure, pass through spinal cord tissue and spinal cord blood vessels, and pass through the second vertebrae structure; and
detecting light passing through the second vertebrae structure using the light detector, and producing sensor signals representative of such detected light; and
processing the sensor signals to obtain data relating to the blood oxygenation level of the subject's spinal cord tissue and spinal cord blood vessels.

9. The method of claim 8, wherein the aligning steps include providing a sensor that includes the light source and the light detector spaced apart from one another by a distance representative of a distance from the first vertebrae to the second vertebrae structure.

10. The method of claim 9, wherein the distance separating the light source from the light detector is based on a distance representative of a distance between first and second vertebrae structures located within a lumbar region of a human spine.

11. The method of claim 9, wherein the distance separating the light source from the light detector is based on a distance representative of a distance between first and second vertebrae structures located within a cervical region of a human spine.

12. The method of claim 9, wherein the distance separating the light source from the light detector is based on a distance representative of a distance between first and second vertebrae structures located within a thoracic region of a human spine.
